(19) United States
(12) Patent Application Publication
(10) Pub. No.: US 2007/0243522 A1
(43) Pub. Date: Oct. 18, 2007

(54) INSPECTION CHIP FOR BIOLOGICAL MATERIAL

(76) Inventors: Yasuhiko Sasaki, Tsuchiura (JP); Toru Inaba, Tokyo (JP); Hiroshi Kishida, Kawasaki (JP); Osamu Kogi, Yokohama (JP); Masaomi Uchida, Yokohama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/679,214

(22) Filed: Feb. 27, 2007

(30) Foreign Application Priority Data
Apr. 13, 2006 (JP) 2006-111305

(51) Int. Cl. G01N 33/00; C12M 1/00

(52) U.S. Cl. 435/4; 435/287.1

(57) ABSTRACT

There is provided an inspection chip capable of performing a number of solution feeding processes promptly and accurately. The inspection chip has one continuous flow path comprising a reaction flow path for accommodating a plurality of beads with immobilized probes of types different each other, a first and second solution holding flow path for holding a plurality of solutions each separated by an air gap. The solution is moved from one solution holding flow path to other solution holding flow path via a reaction flow path by utilizing pressure difference.

### BACKGROUND OF THE INVENTION

1. Field of the Invention

The present invention relates to an inspection chip for detecting biological materials such as peptides, proteins, DNAs, RNAs or the like and an inspection chip system using it.

2. Description of the Related Art

Decoding of base sequences of human genome has been completed, and now activities to know living body at DNA level, and to utilize DNA for understanding of life phenomena and for examination of disorders have been taking place actively. To accomplish these activities, it is important to discriminate at one time differences of genotypes and of expression status of genes in the cells for many instances, and to make comparison between each of disorders or individuals. As a powerful method for investigating expression status of genes, probe chips (many probes are sectioned into several types on a solid substance such as glass slide), DNA chips and further, protein chips are used.

For technologies used to produce such chips, one method is to synthesize oligomers with designed sequence onto many cells being sectioned on a glass slide in one-base by one-base fashion using photochemical reactions and lithography technology utilized extensively in the semiconductor industries (Science 251, 767-773 (1991)), and another method is to implant several types of probes on each zone one by one (Anal. Chem. 69, 543-551 (1997)).

Meanwhile, such a method has been proposed that many fine particles (beads) with probes immobilized thereon are prepared, and several types of beads are collected therefrom to produce a biological material inspection chip (Japanese Patent Application Laid-Open No. 11-243997). With this method, since a probe is immobilized by utilizing chemical reaction in the solution, it is possible to produce probe chips with no variation in probe density for every bead. Therefore, high-precision inspection chips can be configured.

### SUMMARY OF THE INVENTION

With the use of the inspection chip described in JP-A No. 11-243997, it is possible to detect many types of DNAs at one time. However, to allow for detection of DNA, many steps including pre-treatment step, hybridization reaction step, washing step or the like should be performed. Moreover, types and numbers of washing solutions used in the washing step are different depending on the sample concerned. Accordingly, many solution feeding processing should be done promptly and accurately.

An object of the present invention is to provide an inspection chip and an inspection chip system capable of performing many solution feeding processing promptly and accurately.

According to the present invention, the inspection chip has one continuous flow path including a reaction flow path for accommodating a plurality of beads on which are immobilized probes each different in types, a first and a second solution holding flow path for holding a plurality of solutions which are separated each other via an air gap.

Making use of pressure difference, a solution is transferred from one solution holding flow path to other solution holding flow path, passing through the reaction flow path.

According to the present invention, many solution feeding processings can be carried out promptly and accurately.

### BRIEF DESCRIPTION OF THE DRAWINGS

FIG. 1 is a schematic view of the biological material inspection system according to the present invention.

FIG. 2 is a drawing showing one example of DNA inspection step.

FIG. 3 is a perspective view showing a flow path in which beads with probes immobilized thereon are arranged.

FIG. 4 is a drawing showing one example of DNA inspection step using the inspection chip according to the present invention.

FIG. 5 is a plan view of the inspection chip according to the present invention.

FIG. 6 is an explanatory drawing showing methods of executing reaction and washing steps using the inspection chip according to the present invention.

FIG. 7 is a drawing showing the configuration of fluid control mechanism of the biological material inspection system in the embodiment of the present invention.

FIG. 8 is a flowchart showing operations of the fluid control mechanism of the biological material inspection chip in the embodiment of the present invention.

### DESCRIPTION OF THE PREFERRED EMBODIMENTS

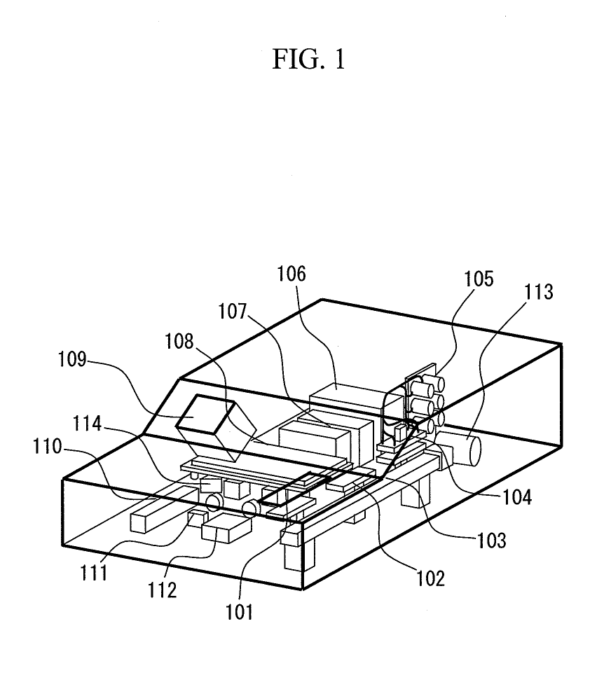

Now, referring to FIG. 1, an embodiment of the biological material inspection system according to the present invention will be explained. The biological material inspection system of the present embodiment has a chip introducing window 101 for insertion of an inspection chip, an optical stage 102 on which the inspection chip is disposed for measuring fluorescence intensity, a transfer stage 103 for transferring the inspection chip, a reaction stage 104 on which the inspection chip is disposed for effecting hybridization reaction, a valve 105 and a pump 113 for solution feeding in the inspection chip, a power supply 106, a motor driver 107, a control substrate 108, an information access panel 109, and an optical system for measuring fluorescence intensity. The optical system includes many optical components such as a laser light source 110, a collecting lens, a mirror 114, a light receiving elements 111, 112, or the like.

The motor driver 107 and the control substrate 108 are used for operations of the transfer stage 103, the valve 105 and pump 113. The power supply 106 supplies electricity to each of various components. The information access panel 109 is used for inputting measuring conditions and outputting measurement results.

The biological material inspection system according to the present invention can detect living body related materials such as DNA, RNA, protein, peptide or the like, while the following description deals with a case for detecting DNA.

First, an inspection chip is inserted from the chip introducing window 101. Beads with immobilized probes are being charged in the inspection chip, and further, sample containing fluorescently-labeled DNA, pre-hybridization solution, washing solution or the like are accommodated. Details of the structure of the inspection chip will be explained later. Next, the inspection chip is transferred to the reaction stage 104 by the transfer stage 103. In the reaction stage 104, the pre-hybridization solution is passed through the beads with probes immobilized thereon in the inspection chip, for initiating pre-hybridization.

Next, a sample solution containing DNA is passed through the beads with probes immobilized thereon to initiate hybridization. By hybridization, DNA fragments in the sample combine with DNA of the probe in a complementary strand fashion. Upon completion of hybridization, the beads are washed with more than one types of washing solutions to remove unreacted DNAs. The pump 113 and valve 105 are used for feeding of the sample solution and washing solution. Details of such solution feeding will be explained later.

Upon completion of the washing, the inspection chip is moved up to the optical stage 102 by the transfer stage 103. In the optical stage 102, a laser from the laser light source 110 is concentrated by a lens and is then irradiated to the probe. Since DNA in the sample captured by the probe is fluorescently-labeled, it emits fluorescence when a laser is irradiated. This fluorescence is wavelength selected by a filter and is detected by an optical detector. As the optical detector, CCD camera and photomultiplier are used. An image obtained by the optical detector is displayed on the information access panel 109.

Beads are arranged along with the flow path in the inspection chip. Probes immobilized on beads are different each other. Therefore, type of the probe is identified according to the position of the bead in the flow path. For the sake of detection of the position of the bead, bead itself may be fluorescently-labeled. APD (avalanche photodiode) which is a light receiving element is used for measurement of fluorescence from the bead. APD separates fluorescence from the bead and fluorescence from the DNA by their wavelength. CCD camera may be used in lieu of APD. CCD camera can detect position of a bead although it does not perform separation by wavelength. PMT (Photomultiplier) which is a light receiving element having sensitivity higher than that of APD may be used. Separation by wavelength is made possible with the use of dichroic mirror.

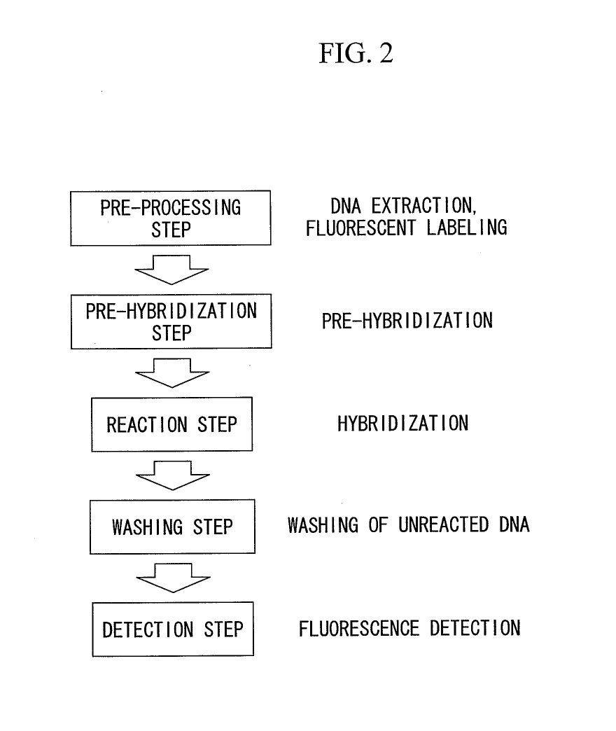

Referring to FIG. 2, outline of procedure for detecting DNA will be explained. The procedure for detecting DNA includes the following five steps: namely, “pre-processing step”, “pre-hybridization step”, “reaction step”, “washing step”, and “detection step”. In the pre-processing step, DNA is extracted from a living body and is fluorescently-labeled. A sample containing DNA is thus prepared. In the pre-hybridization step, pre-hybridization solution and DNA of the probe are subjected to pre-hybridization. In the reaction step, DNA of the sample solution and DNA of the probe are subjected to hybridization. In the washing step, DNAs left unreacted are washed. In the detection step, fluorescence from the DNA captured by the probe is detected.

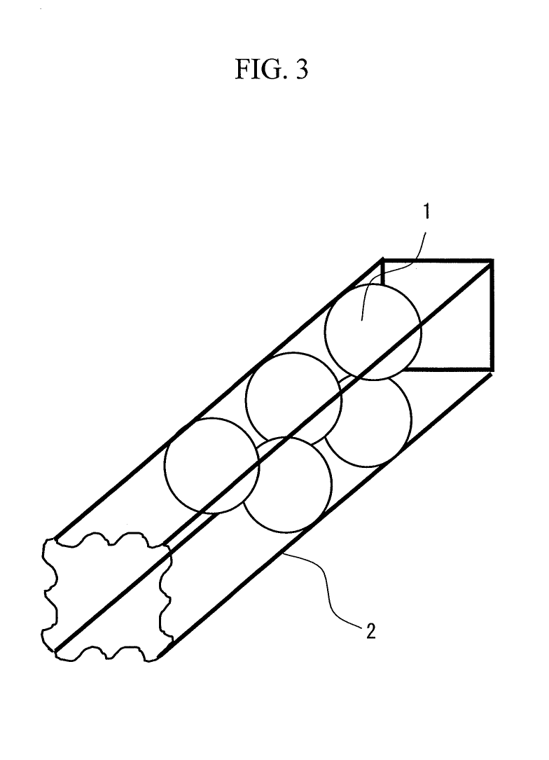

Referring to FIG. 3, beads being charged in the inspection chip will be explained. As illustrated, bead 1 where probes are immobilized is disposed in the reaction flow path 2 being formed in the inspection chip. The method of preparation of the bead 1 is described in JP-A No. 11-243997 and therefore, its explanation will not be described here. Although beads in spherical form are charged in the embodiment illustrated, beads in rectangular form or other configuration may be used. Although dimensions of beads are normally in a range from 1 to 300 microns, beads used in this embodiment have spherical form of 100 microns in size. As for the material of beads, glass or plastics is normally used, while metal such as gold or the like can be employed. In the following explanation, those using glass will be described.

Beads are held in the reaction flow path 2 one-dimensionally, two-dimensionally, or three-dimensionally. FIG. 3 illustrates a case where beads are held three-dimensionally.

The reaction flow path 2 may be a flow path having circular form as represented by a capillary, preferably, may be a flow path composed of PDMS (Polydimethylsiloxane, (C₂H₆SiO)n), which is one sort of silicon resin, formed on a glass substrate. As advantages of using PDMS as material of the flow path, the following three points are mentioned. First, once a die has been produced, formation of a flow path is very simple and at the same time, less expensive. Second, different from the capillary, it is possible to form a flow path with diversified configurations. Namely, a flow path with sophisticated profiles and sections can be formed with ease. Third, optical characteristics are excellent. In other words, since self-fluorescence thereof is very low, error or noise involved in measuring fluorescence intensity of DNA becomes less significant. In the following explanation, it is assumed that the reaction flow path 2 is formed by PDMS. As for materials of the flow path, glass, hard resin and silicon can be used in addition to PDMS.

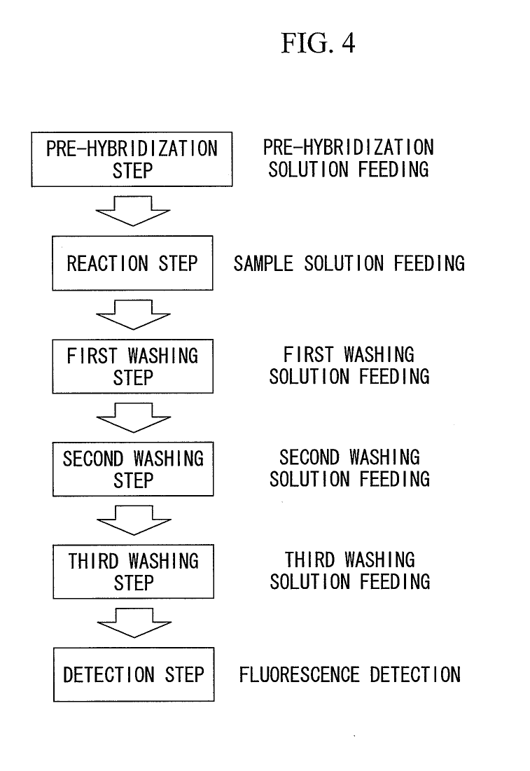

FIG. 4 explains the procedure for detecting DNA using the inspection chip according to the present invention. Here, it is assumed that the pre-processing step has been completed. First, in the pre-hybridization step, pre-hybridization solution is reciprocated in the flow path in which beads with immobilized probes are charged, which effects pre-hybridization. In the next reaction step, a sample solution containing DNA is reciprocated in the flow path in which beads with immobilized probes are charged. Thus, DNA of the sample solution and DNA of the probe are hybridized. In the first to third washing steps, the washing solution is reciprocated in the flow path in which beads with immobilized probes are charged. Thus, unreacted DNAs are washed and removed. Different washing solutions are used in three washing steps. In the detection step, a laser light is irradiated to beads to detect fluorescence emitted from DNA captured by the probe.

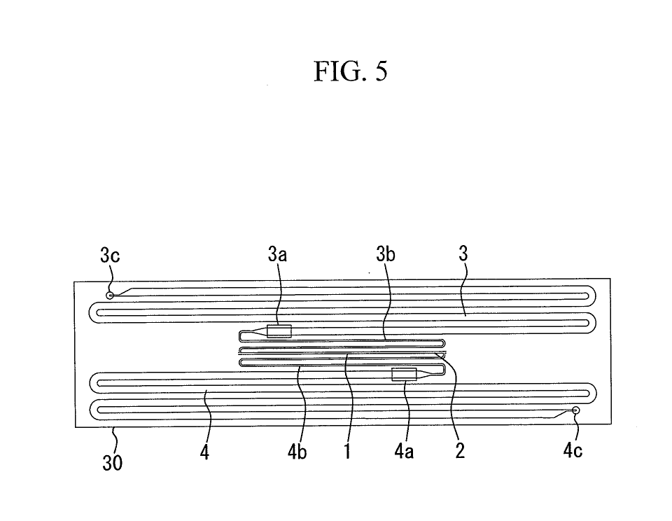

Referring to FIG. 5, structure of an inspection chip 30 according to the present invention will be explained. The inspection chip 30 of the present embodiment has a first transport port 3c, a first solution holding flow path 3, a first air holding flow path 3b, a reaction flow path 2, a second air holding flow path 4b, a second solution holding flow path 4, and a second transport port 4c. The path from the first transport port 3c to the second transport port 4c is formed by one continuous pipe. However, the inside diameter thereof is not constant. For example, the inside diameters of the air holding flow paths 3b, 4b, and the reaction flow path 2 are smaller than the inside diameters of the solution holding flow paths 3, 4. A plurality of beads 1 on which probes of types different each other are immobilized are accommodated in the reaction flow path 2.

The solution holding flow paths 3, 4 accommodate pre-hybridization solution, sample solution and washing solution. The inspection chip of the present embodiment is designed to carry out three washing steps as shown in FIG. 4, and three types of washing solutions are accommodated accordingly in the solution holding flow path. The number of types of washing solutions to be used depends on the inspection object.

Solution detection units 3a, 4a are provided respectively at the inner ends of the solution holding flow paths 3, 4. An optical sensor (not shown) comprising a light emitting unit and a light receiving unit is provided at each of the solution detection units 3a, 4a. Whether or not a solution has passed through each of the solution detection units 3a, 4a is detected by the optical sensor. The solution detection units 3a, 4a are made of transparent material to allow for observation of solution in the flow path.

The air holding flow paths 3b, 4b, are provided for ensuring stabilization of solution feeding between the solution holding flow paths 3, 4 and the reaction flow path 2. Function of the air holding flow paths 3b, 4b will be explained later.

The transport ports 3c, 4c are used when transferring the solution. Solution feeding is performed by applying a high-pressure to either of transport ports 3c, 4c and the other is made open to the atmosphere. Since solution feeding is carried out by utilizing pressure difference, a high-pressure may be applied to one of transport ports 3c, 4c and a low-pressure may be connected to the other. When the inspection chip is mounted on the biological material inspection system shown in FIG. 1, change-over of the transport ports 3c, 4c is performed by shifting the valve 105.

According to the present embodiment, a plurality of different solutions are held at the same time in each of solution holding flow paths 3, 4. An air gap is provided between solutions. By providing an air gap, mixing of adjoining two solutions is prevented. Each solution is held being sandwiched by air gaps at both sides, i.e., fore and aft, moves from the solution holding flow paths 3, 4 to the air holding flow paths 3b, 4b in its entirety, and further moves in the reaction flow path 2. When the air gap moves from the solution holding flow path 3 to the air holding flow path 3b, length of the air gap becomes longer since its inside diameter becomes smaller. When the air gap moves from the reaction flow path 2 to the solution holding flow path 4 via the air holding flow path 4b, length of the air gap becomes shorter.

In the present embodiment, dimensions and diameters of the air holding flow paths 3b, 4b are set so that cubic content of each of air holding flow paths 3b, 4b may become greater than cubic volume of one air gap. Therefore, there is no opportunity that one air gap occupies the air holding flow path 3b, reaction flow path 2, and air holding flow path 4b at one time. Further, dimensions and diameters of the air holding flow paths 3b, 4b are set so that pressure losses of the air holding flow paths 3b, 4b may be substantially identical with pressure loss of the reaction flow path 2.

In general, pressure loss across the reaction flow path 2 is large since beads are charged therein. If pressure losses across the air holding flow paths 3b, 4b are small, a sudden change in pressure loss occurs between the air holding flow paths 3b, 4b and the reaction flow path 2. For example, when a solution enters from the air holding flow path 3b to the reaction flow path 2, an air gap moving together with the solution is compressed. Contrary, when a solution moves from the reaction flow path 2 to the air holding flow path 4b, an air gap moving together with the solution is swollen. Such compression and expansion hinder stable solution feeding. In the present embodiment, sharp change in pressure loss does not take place by the fact that pressure losses across the air holding flow paths 3b, 4b are set to be nearly same as that of across the reaction flow path 2. Thus, stable solution feeding can be realized.

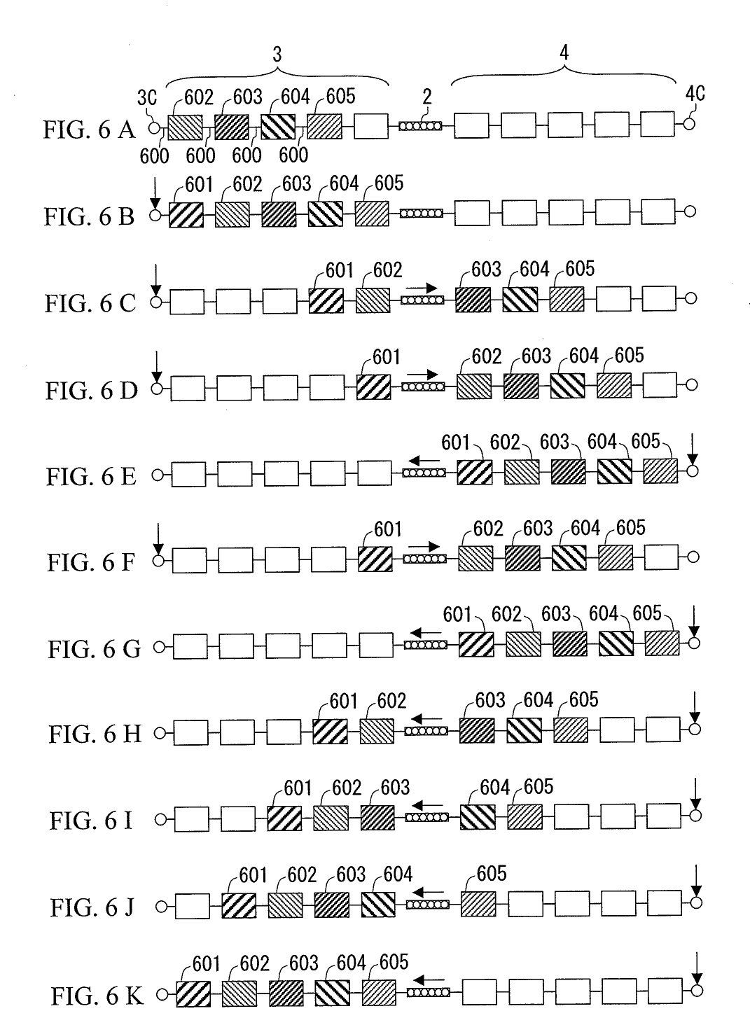

Next, referring to FIG. 6, manipulations of the inspection chip according to the present invention will be explained. FIG. 6 (a) shows a state where a third washing solution 605, a second washing solution 604, a first washing solution 603, and pre-hybridization solution 602 have been charged in this order to the first solution holding flow path 3. The third washing solution 605 is arranged at the side close to the reaction flow path 2 and the pre-hybridization solution 602 is arranged at the side close to the first transport port 3c. An air gap 600 is being inserted between adjoining solutions. Empty boxes in the solution holding flow paths 3, 4 mean that no solution is charged.

Although the user may charge the first solution holding flow path 3 with the solution as mentioned above, an inspection chip to which such solutions are charged in advance may be used.

Next, as shown in FIG. 6 (b), the user charges a sample solution 601 into the first solution holding flow path 3 via the first transport port 3c. On this occasion, the air gap 600 is sandwiched between the sample solution 601 and pre-hybridization solution 602. By charging the sample solution 601, the third washing solution 605, second washing solution 604, first washing solution 603, and pre-hybridization solution 602 are moved in the direction of the reaction flow path 2. In this way, the third washing solution 605, second washing solution 604, first washing solution 603, pre-hybridization solution 602, and the sample solution 601 are charged into the first solution holding flow path 3. However, the air gap 600 is being inserted between adjoining solutions. The inspection chip in the state as shown in FIG. 6 (b) is mounted on the biological material inspection system shown in FIG. 1.

A high-pressure is applied to a first transfer port 3c and a second transfer port 4c is made open to the atmosphere. As a result, as shown in FIG. 6 (c), the third washing solution 605, second washing solution 604, and first washing solution 603 in the first solution holding flow path 3 pass through the reaction flow path 2 in this order, and move into the second solution holding flow path 4. Even when the third washing solution 605, second washing solution 604, and first washing solution 603 move from the first solution holding flow path 3 into the second solution holding path 4 via the reaction flow path 2, the air gap 600 between adjoining solutions move together with solutions as it is. Therefore, there is no opportunity that adjoining solutions are mixed.

First, the pre-hybridization step is executed. The high-pressure is applied to the first transfer port 3c and the second transfer port 4c is made open to the atmosphere. As shown in FIG. 6 (d), the pre-hybridization solution 602 in the first solution holding flow path 3 passes through the reaction flow path 2. This operation results in occurrence of pre-hybridization. The pre-hybridization solution 602 passed through the reaction flow path 2 moves into the second solution holding flow path 4.

Next, the reaction step is executed. The high-pressure is applied to the first transfer port 3c and the second transfer port 4c is made open to the atmosphere. The sample solution 601 in the first solution holding flow path 3 passes through the reaction flow path 2. Thus, DNA of the sample solution and DNA of the probe are hybridized. The sample solution 601 passed through the reaction flow path 2 moves into the second solution holding flow path 4.

Next, as shown in FIG. 6 (e), the high-pressure is applied to the second transfer port 4c and the first transfer port 3c is made open to the atmosphere. As a result, the sample solution 601 in the second solution holding flow path 4 passes through the reaction flow path 2. Thus, DNA of the sample solution and DNA of the probe are hybridized. Next, as shown in FIG. 6 (f), the high-pressure is applied to the first transfer port 3c and the second transfer port 4c is made open to the atmosphere. As a result, the sample solution 601 in the first solution holding flow path 3 passes through the reaction flow path 2. Thus, DNA of the sample solution and the probe immobilized on the bead are hybridized. In this way, the sample solution 601 is reciprocated in the reaction flow path 2 a predetermined number of times.

As shown in FIG. 6 (g), while the third washing solution 605, second washing solution 604, first washing solution 603, pre-hybridization solution 602, and the sample solution 601 are being charged in the second solution holding flow path 4, the high-pressure is applied to the second transport port 4c, and the first transport port 3c is made open to the atmosphere. First, the sample solution 601 in the second solution holding flow path 4 passes through the reaction flow path 2 and last hybridization is performed. Next, the pre-hybridization solution 602 in the second solution holding flow path 4 passes through the reaction flow path 2. Thus, as shown in FIG. 6 (h), the sample solution 601 in the second solution holding flow path 4 and the pre-hybridization solution 602 move into the first solution holding flow path 3.

Next, the washing step is executed. The high-pressure is applied to the second transfer port 4c and the first transfer port 3c is made open to the atmosphere. As shown in FIG. 6 (i), the first washing solution 603 in the second solution holding flow path 4 passes through the reaction flow path 2 and moves into the first solution holding flow path 3. In this way, the first washing step is carried out. Similarly, the high-pressure is applied to the second transfer port 4c and the first transfer port 3c is made open to the atmosphere. As shown in FIG. 6 (j), the second washing solution 604 in the second solution holding flow path 4 passes through reaction flow path 2 and moves into the first solution holding flow path 3. In this way, the second washing step is executed. The high-pressure is applied to the second transfer port 4c and the first transfer port 3c is made open to the atmosphere. As shown in FIG. 6 (k), the third washing solution 605 in the second solution holding flow path 4 passes through the reaction flow path 2 and moves into the first solution holding flow path 3. In this way, the third washing step is executed. Unreacted DNAs adhered to the beads are washed away by executing the first, second and third washing steps as mentioned above. Upon completion of the washing steps, the inspection step is executed.

In FIG. 6, the air holding flow paths 3b, 4b are not shown. However, in practice, when the solution in the first solution holding flow path 3 moves into the second solution holding flow path 4 via the reaction flow path 2, it passes through the air holding flow paths 3b, 4b. Contrary, when the solution in the second solution holding flow path 4 moves into the first solution holding flow path 3 via the reaction flow path 2, it passes through the air holding flow paths 3b, 4b.

When a solution moves, air gaps before and after the solution also move. According to the present embodiment, cubic contents of the air holding flow paths 3, 4 are greater than volume of the air gap. Therefore, there is no opportunity that one air gap occupies the air holding flow paths 3, 4 and reaction flow path 2 at one time.

Further, pressure losses of the air holding flow paths 3b, 4b are set to be nearly identical with pressure loss of the reaction flow path 2. According to the present embodiment, since no sudden change in pressure loss occurs, compression of air gap moving from the air holding flow paths 3b, 4b into the reaction flow path 2, and expansion of air gap moving from the reaction flow path 2 to the air holding flow paths 3b, 4b can be prevented. Therefore, solution feeding can be performed in stable fashion.

Further, by inserting an air gap between adjoining solutions, detection of solution feeding at the solution detection units 3a, 4a can be made with ease. For example, it is possible to detect solution feeding by utilizing a difference of optical transmissivity or reflectance between liquid such as sample solution and gas such as air gap.

According to the present embodiment, upon completion of the inspection step, the inspection chip is discarded without modification. In other words, waste solutions of sample solution, hybridization solution and washing solution are discarded being maintained in the inspection chip. Therefore, disposal of these waste solutions can be made safely and simply.

According to the present embodiment, by simply applying the high pressure to one of a pair of transfer ports and by opening the other to the atmosphere, all solutions in the inspection chip can be fed one after another. Accordingly, there is no need for a movement mechanism for moving the inspection chip, which allows for reduced size biological material inspection chip system.

According to the embodiment shown in FIG. 6, the sample solution 601, pre-hybridization solution 602, and washing solution 603, 604, 605 are transferred from the first solution holding flow path 3 to the second solution holding flow path 4, and then returned them to the first solution holding flow path 3. Namely, reciprocating solution feeding is performed. Meanwhile, solution feeding in one direction only may be used, as necessary. For example, the pre-hybridization solution 602, sample solution 601, first washing solution 603, second washing solution 604, third washing solution 605 are charged into the first solution holding flow path 3 in this order. The pre-hybridization solution 602 is arranged closest to the reaction flow path 2 and the third washing solution 605 is arranged farthest away from the reaction flow path 2. Next, the high-pressure is applied to the first transport port 3c and the second transport port 4c is made open to the atmosphere. First, the pre-hybridization solution 602 passes through the reaction flow path 2 and moves to the second solution holding flow path 4. Next, the sample solution 601 passes through the reaction flow path 2 and moves to the second solution holding flow path 4. Similarly, the first washing solution 603, second washing solution 604, and third washing solution 605 pass through the reaction flow path 2 in series and move to the second solution holding flow path 4. In this way, hybridization can be carried out by solution feeding in one direction.

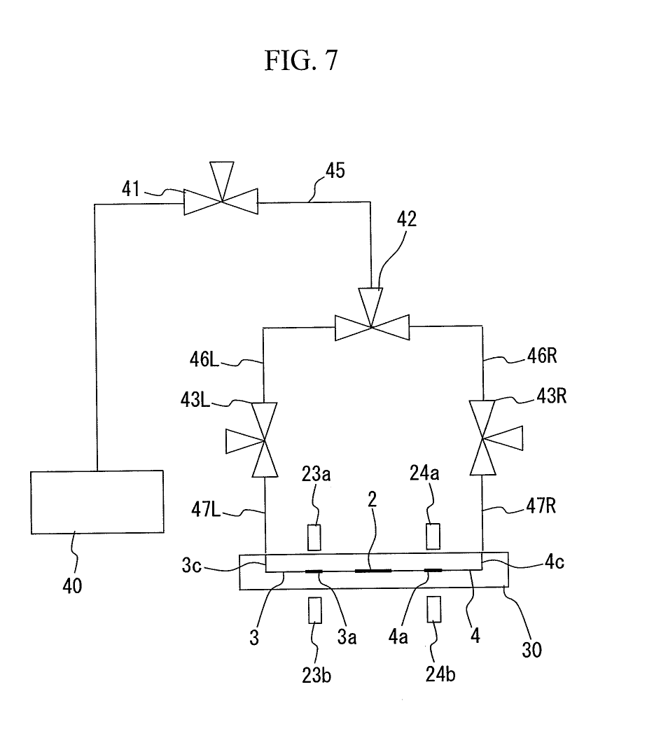

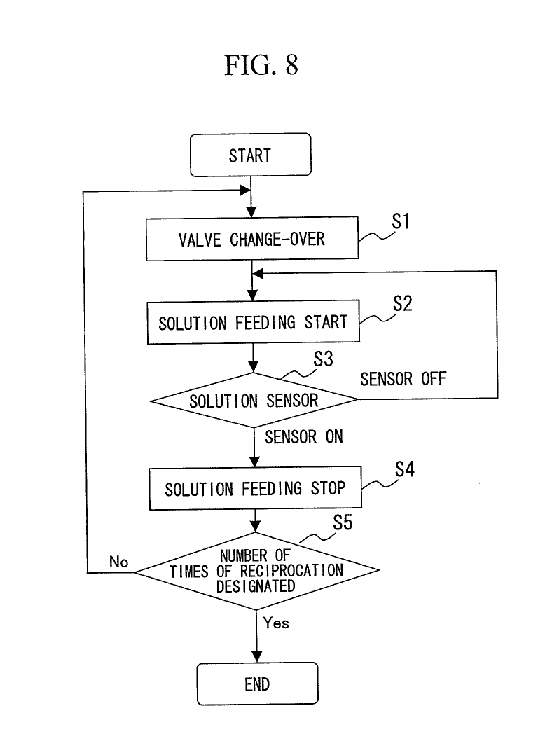

Now, explanation will be given referring to FIG. 7 and FIG. 8. FIG. 7 shows outline of a fluid control mechanism of the biological material inspection chip system of the present embodiment, and FIG. 8 shows flowchart of operations of the fluid control mechanism. A case where hybridization, which is explained using FIG. 6, is carried out by using the fluid control mechanism will be explained here. As shown in FIG. 7, the fluid control mechanism of the present embodiment has a pressure source 40, valves 41, 42, 43L, 43R, pipes 45, 46L, 46R, 47L, 47R. In FIG. 7, the reaction flow path 2, solution holding flow paths 3, 4, transport ports 3c, 4c, and solution detection units 3a, 4a alone in the inspection chip 30 are illustrated schematically. At upper portion of the solution detection units 3a, 4a in the inspection chip 30 are disposed light emitting units 23a, 24a and at lower portion, light receiving units 23b, 24b are disposed, respectively.

During solution feeding, the valve 41 connects the pressure source 40 to the pipe 45. The valve 42 connects the pipe 45 to either of pipes 46L, 46R. The valve 43L connects two pipes 46L, 47L each other or connects the both to the atmosphere. The valve 43R connects two pipes 46R, 47R each other or connects the both to the atmosphere. The pipe 47L is connected to the first transport port 3c and the pipe 47R is connected to the second transport port 4c.

First, solution feeding in outward direction is performed. As shown in FIG. 8, the valve is changed-over in step S1. The pipe 45 is connected to the pipe 46L by the valve 42, and the pipe 46L is connected to the pipe 47L by the valve 43L. Thus, the pressure source 40 is connected to the first transport port 3c. The pipes 46R, 47R are made open to the atmosphere by the valve 43R. As a result, the second transport port 4c is connected to the atmosphere.

In step S2, solution feeding is started. A pressure from the pressure source 40 is applied to the first transport port 3c via the pipes 45, 46L, 47L. As a result, as shown in FIG. 6 (c), the third washing solution 605, second washing solution 604, and first washing solution 603 in the solution holding flow path 3 pass through the reaction flow path 2 in series and move into the second solution holding flow path 4.

In step S3, a solution sensor determines whether or not all solutions have passed through the solution detection unit 3a, 4a. When all solutions have not passed through, it returns to step S2, and remaining solutions, i.e., pre-hybridization solution 602 and sample solution 601 are fed. After all solutions have passed through, it proceeds to step S4 and solution feeding is stopped. The pipes 46L, 47L are made open to the atmosphere by change-over of the valve 43L. As a result, the first transport port 3c is made open to the atmosphere, and solution feeding in outward direction is performed.

In step S5, it is determined whether or not a number of times of reciprocation designated is being set. When the number of times of reciprocation designated is not set, the processing is terminated. When the number of times of reciprocation designated is set, it returns to step S1.

In step S1, change-over of the valve is performed. The pipe 45 is connected to the pipe 46R by the valve 42, and the pipe 46R is connected to the pipe 47R by the valve 43R. Thus, the pressure source 40 is connected to the second transport port 4c. The pipes 46L, 47L are made open to the atmosphere by the valve 43L. As a result, the first transport port 3c is connected to the atmosphere. In step S2, solution feeding is started. A pressure from the pressure source 40 is applied to the second transport port 4c via the pipes 45, 46R, 47R. As a result, as shown in FIG. 6 (e) and FIG. 6 (f), the sample solution in the second solution holding flow path 4 passes through the reaction flow path 2 and moves into the first solution holding flow path 3.

In step S3, the solution sensor determines whether or not the sample solution has passed the solution detection unit 4a. When the sample solution has not passed through, it returns to step S2 and continues solution feeding. When the sample solution has passed through, it proceeds to step S4 and stops solution feeding. The pipes 46R, 47R are made open to the atmosphere by change-over of the valve 43R. As a result, solution feeding in inward direction is performed. When, in step S5, reciprocation is carried out as many as the number of times of reciprocation designated, the processing is terminated.

In the present embodiment, solution feeding is controlled while determination is made by the solution sensor whether or not the solution has passed the solution detection unit. Therefore, it is possible to carry out hybridization accurately without observing a solution in the inspection chip by human.

As mentioned above, according to the present invention, in the inspection chip using beads with immobilized probes, the solution detection unit is provided in the flow path, and fluid control is carried out by detecting passing of solutions such as sample, washing solution, or the like. Therefore, fluid control of solutions can be carried out accurately in the inspection chip, thereby improving stability of amount of sample reaction and amount of washing in the chip.

Although embodiments of the present invention are explained above, the present invention is not limited thereto, and it will readily be understood by those skilled in the art that various modifications may be made within the scope of the invention according to the claims.

What is claimed is:

1. An inspection chip having one continuous flow path, the flow path comprising a reaction flow path for accommodating a plurality of beads with immobilized probes of types different each other, a first and a second solution holding flow paths each for holding a plurality of solutions each being separated via an air gap, the inspection chip being configured as such that the solution is moved, by utilizing a pressure difference, from one of the first and second solution holding flow paths, passing through the reaction flow path, to the other of the first and second solution holding flow paths.

2. The inspection chip according to claim 1, wherein a first and a second air holding flow paths are provided between the reaction flow path, and the first and second solution holding flow paths, respectively, and pressure losses of the air holding flow paths are nearly identical with a pressure loss of the reaction flow path.

3. The inspection chip according to claim 2, wherein cubic contents of the air holding flow paths are greater than cubic volume of the air gap.

4. The inspection chip according to claim 1, wherein a transport port is provided at each end of the flow paths, and the pressure difference is generated by applying a high-pressure to one of the transport ports and a low-pressure is applied to the other.

5. The inspection chip according to claim 1, wherein a washing solution and a pre-hybridization solution are being charged to one of the first and second solution holding flow paths.

6. The inspection chip according to claim 1, wherein the sample solution charged by a user is held in one of the solution holding flow paths.

7. The inspection chip according to claim 1, wherein at least a part of the flow paths is formed by PDMS (polydimethylsiloxane).

8. An inspection chip system comprising: an inspection chip having one continuous flow path comprising a reaction flow path for accommodating a plurality of beads with immobilized probes of types different each other, a first and a second solution holding flow paths each for holding a plurality of solutions each being separated via an air gap; a solution detection apparatus for detecting whether or not the solution is fed to the reaction flow path; a pressure source; a control apparatus for connecting to the inspection chip a pressure from the pressure source; wherein the control apparatus is configured as such that the solution is moved, by utilizing a pressure difference generated by a pressure from the pressure source, on the basis of a solution detection signal detected by the solution detection apparatus, from one of the first and second solution holding flow paths, passing through the reaction flow path, to the other of the first and second solution holding flow paths.

9. The inspection chip system according to claim 8, wherein a washing solution, a pre-hybridization solution and a sample solution are moved in this order from the first solution holding flow path, passing through the reaction flow path, to the second solution holding flow path, and then the sample solution, pre-hybridization solution and washing solution are moved in this order from the second solution holding flow path, passing through the reaction flow path, to the first solution holding flow path.

10. The inspection chip system according to claim 8, wherein the pre-hybridization solution, sample solution and washing solution are moved in this order from the first solution holding flow path, passing through the reaction flow path, to the second solution holding flow path.

11. A hybridization experimental methodology for performing hybridization using an inspection chip having one continuous flow path comprising a reaction flow path for accommodating a plurality of beads with immobilized probes of types different each other, a first and a second solution holding flow paths provided at respective ends of the reaction flow path, the inspection chip being configured so as to perform solution feeding utilizing a pressure difference, the hybridization experimental methodology comprising:
a step of arranging a washing solution, a pre-hybridization solution and a sample solution in this order from near the reaction flow path so as to be separated each other via air gaps in the first solution holding flow paths;
a step of moving the washing solution, the pre-hybridization solution and the sample solution in this order in an forward path which is from the first solution holding flow path, via the reaction flow path, to the second solution holding flow path; and
a step of moving the sample solution, the pre-hybridization solution, and the washing solution in this order in an backward path which is from the second solution holding flow path, via the reaction flow path, to the first solution holding flow path.

12. The hybridization experimental methodology according to claim 11 further comprising, between the step of moving solutions in the forward path and the step of moving solutions in the backward path:
a step of moving the sample solution from the second solution holding flow path, via the reaction flow path, to the first solution holding flow path; and
a step of moving the sample solution from the first solution holding flow path, via the reaction flow path, to the second solution holding flow path.

13. A hybridization experimental methodology for performing hybridization using an inspection chip having one continuous flow path comprising a reaction flow path for accommodating a plurality of beads with immobilized probes of types different each other, a first and a second solution holding flow paths provided at respective ends of the reaction flow path, the inspection chip being configured so as to perform solution feeding utilizing a pressure difference, the hybridization experimental methodology comprising:
a step of arranging a pre-hybridization solution, a sample solution and a washing solution in this order from near the reaction flow path so as to be separated each other via air gaps in the first solution holding flow path; and
a step of moving the pre-hybridization solution, the sample solution and the washing solution in this order from the first solution holding flow path, via the reaction flow path, to the second solution holding flow path.
